(19) United States
(12) Patent Application Publication
(10) Pub. No.: US 2011/0218354 A1
(43) Pub. Date: Sep. 8, 2011

(54) Photoautotrophic Adipogenesis Technology (Phat)

(75) Inventors: David Savage, Cambridge, MA (US); Pamela Silva, Cambridge, MA (US)

(73) Assignee: President and Fellows of Harvard College, Cambridge, MA (US)

(21) Appl. No.: 13/119,918

(22) PCT Filed: Sep. 21, 2009
(86) PCT No.: PCT/US2009/057694

§ 371 Date: May 20, 2011

Related U.S. Application Data
(60) Provisional application No. 61/192,663, filed on Sep. 19, 2008.

(51) Int. Cl. C07C 69/00; C12N 1/00; C12N 1/21; C12P 5/00

(52) U.S. Cl. 560/129; 435/243; 435/252.3; 435/166

(57) ABSTRACT

The invention provides an isolated photosynthetic microorganism with an altered lipid profile compared to its wild type or naturally occurring counterpart. The bacterium has been genetically modified such that the lipids produced are amenable to producing biodiesel fuel. Accordingly, such an isolated cyanobacterium contains a fatty acid, the length of which is less than C 16.

### FIELD OF THE INVENTION

The invention relates to production of biofuels.

### BACKGROUND OF THE INVENTION

Biofuels are fuels that come from biological origin like plants, animals, or bacteria. Some bacteria capture energy in the form of sunlight and use it to carry out a variety of biochemical processes. Photosynthesis provides a mechanism for capturing solar energy to directly drive a myriad of chemical processes. After light energy is captured, it is converted to chemical energy and precursors in the form of carbohydrates through a series of enzymatic steps. Conversion of carbohydrate into useful molecules including fuels such as ethanol, butanol, or biodiesel also proceeds through specific enzymatic steps. It would be a major advance to construct microbes that efficiently harness photosynthesis to drive the biosynthesis of useful chemicals such as biofuels.

### SUMMARY OF THE INVENTION

The invention provides an isolated photosynthetic microbe with an altered lipid profile compared to its wild type or naturally occurring counterpart. For example, the photosynthetic microbe is a such as a cyanobacterium or blue-green algae, e.g., Synechococcus sp. The bacterium has been genetically modified such that the lipids produced are amenable to producing biodiesel fuel. Accordingly, such an isolated cyanobacterium contains a fatty acid, the length of which is less than C16. At least 10% of the lipids in the microbe are less than 16 carbons (16C) in length, preferably, at least 25%, 50%, 75%, 90%, 95%, 99%, and up to 100% of the lipids are less than 16 carbons in length. For example, the chain length is 15, 14, 13, 12, 11, 10, 9, 8, or less. In a preferred embodiment, the length is C12, e.g., the fatty acid fatty acid comprises lauric acid (C₁₁H₂₃COOH). The microbes are used to produce a combustible biofuel.

To achieve an altered lipid profile, the bacterium contains an enzyme derived from a species other that that of the host species. For example, the cyanobacterium contains a heterologous short or medium-chain acyl-acyl carrier protein thioesterase enzyme such as California bay tree thioesterase (CBT), I.E. FatB1 from Cuphea palustris (as well as all medium-chain specific enzymes from the genus Cuphea) or luxD from Vibrio harveyi. An exemplary amino acid sequence is provided in SEQ ID NO:9 or a fragment thereof, and an exemplary nucleic acid sequence encoding the enzyme is SEQ ID NO:6 or a fragment thereof. Bacteria genetically altered in this manner produce shorter lipids compared to wild type or naturally-occurring bacteria. These shorter fatty acids are better suited to the production of biodiesel fuel, because unlike longer fatty acids that freeze or become viscous at low temperatures, these fatty acids have a higher cloud or gel point. Thus, the thioesterase-modified bacteria are a better source of fatty acids from which to make biodiesel fuel. A method of producing a biodiesel fuel is carried out by culturing the thioesterase-modified bacterium and extracting a medium chain fatty acid from the bacterium and/or culture medium using conventional chemical techniques, e.g., Huber et al., Chem Rev. 2006 September; 106(9):4044-98, for producing a biodiesel fuel. For example, the fatty acids obtained from the bacteria are esterified to yield a medium chain hydrocarbon biodiesel fuel composition.

In an alternate approach, a single bacterium is used to produce biodiesel fuel. In that case, the bacterium is engineered to express additional heterologous proteins that are required to further process the medium chain fatty acids in the bacterium itself. Biodiesel synthesis is carried out by expression of elements of the Zymomonas mobilis ethanol fermentation pathway and the non-specific acyltransferase of Acinetobacter baylyi, which esterifies fatty acids with ethanol. Accordingly, the cyanobacterium further comprises a heterologous wax synthase such as one containing the amino acid sequence of SEQ ID NO:7 (Wax ester synthase/acyl-CoA:diacylglycerol acyltransferase; WS/DGAT) or a fragment thereof. For example, the enzyme is produced by a nucleic acid comprising SEQ ID NO:8 or a fragment thereof. Other suitable wax synthase enzymes include mammalian (human or mouse) wax synthases (e.g., described in Cheng et al., J. Biol. Chem., Vol. 279, Issue 36, 37798-37807, Sep. 3, 2004; GenBank™ AY605053 (human) or AY611031 and AY611032 (mouse)) as well as Jojoba (Simmondsia chinensis) Synthase (e.g., described in Lardizabal et al., Plant Physiol, March 2000, Vol. 122, pp. 645-656; GenBank™ AB015479).

The cyanobacterium may also comprise a nucleic acid encoding a heterologous pyruvate decarboxylase (PDC). An exemplary amino acid sequence is from Zymomonas mobilis, SEQ ID NO:1 or a fragment thereof, and an exemplary nucleic acid sequence encoding the enzyme includes SEQ ID NO:2 or a fragment thereof. PDCs, e.g., PDC 1, 2, 5 or 6 from Saccharomyces cerevisiae (PDC1, GenBank™ NP_{—}01314.1; PDC5, GenBank™, NP_{—}013235.1; PDC6, GenBank™ NP_{—}011601.1).

The bacterium may further include a heterologous alcohol dehydrogenase (ADHE) such as those including the amino acid sequence of SEQ ID NO:3 (from Zymomonas mobilis) or a fragment thereof. An exemplary nucleic acid comprises SEQ ID NO:4 or a fragment thereof. Other suitable ADHE enzymes include ADH4 or ADH7 from Saccharomyces cerevisiae (ADH4, GenBank™ NP_{—}011258.1; ADH7, GenBank™ NP_{—}010030.1).

For example, the isolated cyanobacterium contains a nucleic acid encoding a heterologous thioesterase, wax synthase, pyruvate decarboxylase, and alcohol dehydrogenase, and a method of producing a biodiesel fuel is carried out by culturing the bacterium and extracting from the bacterium a medium chain hydrocarbon biodiesel fuel composition. Thus, the photosynthetic microbes described herein are useful to produce biodiesel, or a component or intermediate thereof, e.g., laurate ethyl ester, in a solar-powered manner utilizing atmospheric CO₂ as the carbon source.

The invention provides isolated polynucleic acids containing genes or any fragment thereof. Isolated polynucleic acids of the invention are bound, conjugated to, incorporated into, or associated with gene delivery systems for insertion into host cells. Exemplary gene delivery systems include, but are not limited to, polymers, nanoparticles (gold, magnetic), lipids, liposomes, microspheres, proteins, and compounds. Furthermore, isolated polynucleic acids of the invention comprise genes or any fragment thereof and expression vectors, plasmids, markers, reporter genes, enhancers, promoters, repressors, recombination sites (such as loxP or Frt), and any other sequence used to insert or delete genes from engineered yeast and host cells of the invention.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

Other features, objects, and advantages of the invention will be apparent from the description and drawings. All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same.

### BRIEF DESCRIPTION OF THE DRAWINGS

FIG. 1 is a diagram of a nucleic acid construct for an organism that has been engineered to express a heterologous thioesterase.

FIG. 2 is a diagram of a nucleic acid construct for an organism that has been engineered to express a heterologous PDC, heterologous ADHE, and a heterologous WSDGAT.

FIG. 3 is a photograph of the results of a Western blot assay showing thioesterase expression in Synechococcus. Two thioesterase FatB genes were expressed.

FIG. 4 is a line graph showing engineered fatty acid production in stratin 651S expressing U. californica thioesterase. Fatty acid analysis of purified Synechococcus lipids shows that expression of the U. californica thioesterase alters the lipid profile and produces the shorter C12 fatty acid.

FIG. 5 is a bar graph showing ethanol synthesis in Synechococcus using the Zymomonas pdc/adh pathway. Demonstration that ethanol synthesis is accomplished by expression of two genes form Zymomonas.

### DETAILED DESCRIPTION

Liquid transportation fuels (e.g. ethanol or petroleum-based fuels) ultimately derive their energy from the photosynthetic conversion of sunlight into chemical energy. Earlier plant-based strategies have been plagued by the low efficiency with which sunlight is converted into useful molecules within the plant and in further ex vivo processing steps. To overcome this problem, photosynthetic microbes that more efficiently convert light into chemical energy were engineered to direct the chemienergetic flux of photosynthesis into optimized lipids and lipid derivatives. These molecules are harvested and used as a combustible fuel with little to no processing. Thus, the microbes described herein convert sunlight directly into biofuels. The methods permit harnessing and converting of solar energy into chemical energy in a single organism, thereby producing combustible fuels from sunlight.

An exemplary microbe is a photosynthetic cyanobacterium that produces a short alkyl chain by commandeering the flow of electrons/reducing equivalents from Photosystem I into an artificial metabolic pathway. For example, a cyanobacterium is engineered to produce lauric acid ethyl ester (C₁₁H₂₃COOC₂H₅) by forced regulated expression of one or more of the following four proteins: the California bay thioesterase, a wax synthase, pyruvate decarboxylase, and alcohol dehydrogenase. Described below are exemplary nucleic acid and amino acid sequences for each enzyme.

Zymomonas mobilis
Pyruvate decarboxylase
Swissprot ID: P06672
>sp|P06672|PDC_ZYMMO Pyruvate decarboxylase
OS = Zymomonas mobilis GN = pdc PE = 1 SV = 1
(SEQ ID NO: 1)
MSYTVGTYLAERLVQIGLKHHFAVAGDYNLVLLDNLLLNKNMEQVYCC
NELNCGFSAEGYARAKGAAAAVVTYSVGALSAFDAIGGAYAENLPVIL
ISGAPNNNDHAAGHVLHHALGKTDYHYQLEMAKNITAAAEAIYTPEEA
PAKIDHVIKTALREKKPVYLEIACNIASMPCAAPGPASALFNDEASDE
ASLNAAVEETLKFIANRDKVAVLVGSKLRAAGAEEAAVKFADALGGAV
ATMAAAKSFFPEENPHYIGTSWGEVSYPGVEKTMKEADAVIALAPVFN
DYSTTGWTDIPDPKKLVLAEPRSVVVNGIRFPSVHLKDYLTRLAQKVS
KKTGALDFFKSLNAGELKKAAPADPSAPLVNAEIARQVEALLTPNTTV
IAETGDSWFNAQRMKLPNGARVEYEMQWGHIGWSVPAAFGYAVGAPER
RNILMVGDGSFQLTAQEVAQMVRLKLPVIIFLINNYGYTIEVMIHDGP
YNNIKNWDYAGLMEVFNGNGGYDSGAGKGLKAKTGGELAEAIKVALAN
TDGPTLIECFIGREDCTEELVKWGKRVAAANSRKPVNKLL

Codon Optimized Sequence for S. elongatus:

(SEQ ID NO: 2)
ATGTCATATACTGTCGGTACTTATCTAGCTGAACGCTTGGTGCAAAT
TGGTCTGAAACACCACTTTGCAGTGGCAGGGGATTACAACCTAGTGC
TCTTGGATAACCTTTTGTTGAACAAAAACATGGAACAAGTGTATTGT
TGCAATGAGCTAAACTGTGGCTTTTCGGCGGAAGGGTACGCACGCGC
GAAGGGTGCTGCGGCGGCAGTCGTCACCTACTCTGTCGGCGCACTCT
CTGCGTTTGATGCTATCGGTGGCGCTTATGCGGAGAATCTCCCTGTC
ATCCTCATCTCCGGTGCGCCAAACAATAACGACCACGCTGCTGGGCA
CGTCCTCCACCATGCTCTCGGTAAAACAGATTATCATACCAACTTGA
GATGGCGAAAAACATAACCGCTGCTGCGGAGGCGATCTATACTCCAG
AAGAAGCGCCCGCAAAAATCGACCATGTAATTAAGACGGCATTGCGC
GAGAAAAAGCCGGTTTATCTCGAAATTGCTTGCAATATTGCTTCAAT
GCCCTGTGCTGCGCCCGGTCCTGCGTCGGCATTGTTCAACGATGAAG
CAAGCGATGAAGCAAGCCTGAACGCGGCGGTTGAAGAGACGCTGAAA
TTCATTGCAAACAGAGACAAAGTTGCTGTGTTGGTTGGTTCCAAGCT
GCGCGCTGCTGGGGCGGAAGAAGCGGCGGTGAAGTTTGCTGATGCTT
TGGGCGGAGCAGTGGCAACAATGGCTGCTGCTAAATCCTTTTTCCCT
GAGGAGAACCCACATTATATCGGCACCAGCTGGGGAGAGGTAAGCTA
TCCCGGCGTGGAAAAGACGATGAAAGAAGCGGACGCGGTGATTGCTC
TAGCTCCCGTTTTTAATGATTATTCCACGACGGGATGGACTGACATT
CCTGACCCCAAGAAACTGGTTTTGGCGGAACCTCGCAGCGTGGTTGT
GAACGGCATTCGATTCCCAAGTGTGCACCTGAAGGATTACCTCACCC
GCTTAGCTCAAAAAGTCAGCAAGAAGACCGGTGCGCTAGATTTCTTT
AAATCGCTGAACGCGGGTGAACTCAAGAAAGCTGCTCCGGCTGACCC
TTCTGCGCCCCTCGTGAACGCAGAAATCGCTCGTCAAGTGGAGGCGC
TGCTGACGCCGAATACGACTGTTATTGCTGAGACGGGCGATAGCTGG
TTTAATGCTCAGCGAATGAAACTGCCCAACGGGGCTCGCGTGGAATA
TGAAATGCAATGGGGCCATATTGGATGGTCTGTGCCTGCGGCTTTTG
GTTACGCAGTTGGTGCACCCGAACGGCGTAACATCTTGATGGTGGGG
GATGGGAGCTTCCAGCTTACCGCACAAGAGGTGGCTCAAATGGTGCG
CCTGAAATTGCCCGTGATTATCTTCCTGATTAACAATTACGGGTACA
CGATTGAAGTCATGATTCATGATGGCCCCTATAACAACATCAAGAAT
TGGGACTATGCTGGTCTGATGGAAGTCTTTAATGGTAACGGTGGCTA
CGATAGTGGCGCTGGGAAGGGCCTCAAGGCAAAGACGGGTGGGGAAC
TAGCGGAAGCAATTAAAGTCGCGTTGGCAAACACTGACGGGCCGACG
CTCATCGAATGTTTCATCGGTCGCGAGGATTGTACCGAAGAGCTTGT
GAAATGGGGGAAGCGGGTTGCTGCTGCGAATAGTCGTAAGCCGGTGA
ACAAACTGTTG

Zymomonas mobilis
Alcohol dehydrogenase
Swissprot ID: P06758
>sp|P06758|ADH2_ZYMMO Alcohol dehydrogenase 2
OS = Zymomonas mobilis GN = adhB PE = 1 SV = 3
(SEQ ID NO: 3)
MASSTFYIPFVNEMGEGSLEKAIKDLNGSGFKNALIVSDAFMNKSGV
VKQVADLLKAQGINSAVYDGVMPNPTVTAVLEGLKILKDNNSDFVIS
LGGGSPHDCAKAIALVATNGGEVKDYEGIDKSKKPALPLMSINTTAG
TASEMTRFCIITDEVRHVKMAIVDRHVTPMVSVNDPLLMVGMPKGLT
AATGMDALTHAFEAYSSTAATPITDACALKAASMIAKNLKTACDNGK
DMPAREAMAYAQFLAGMAFNNASLGYVHAMAHQLGGYYNLPHGVCNA
VLLPHVLAYNASVVAGRLKDVGVAMGLDIANLGDKEGAEATIQAVRD
LAASIGIPANLTELGAKKEDVPLLADHALKDACALTNPRQGDQKEVE
ELFLSAF

### Nucleic Acid Sequence:

(SEQ ID NO: 4)
ATGGCGTCCAGTACCTTCTACATCCCTTTTGTCAATGAAATGGGTGA
GGGCAGCTTGGAAAAAGCGATCAAGGACCTGAACGGGTCGGGCTTTA
AAAACGCATTGATCGTTAGCGATGCGTTTATGAACAAGAGCGGCGTC
GTCAAACAAGTGGCAGATCTGCTGAAGGCGCAGGGGATCAATTCGGC
GGTTTATGACGGTGTCATGCCCAACCCCACCGTTACTGCTGTCTTGG
AAGGCTTGAAAATTCTCAAGGATAACAACTCTGATTTTGTCATTTCC
CTGGGCGGGGGATCGCCCCATGACTGCGCTAAAGCAATCGCGTTGGT
CGCTACCAACGGCGGTGAGGTTAAAGATTATGAGGGCATCGACAAGT
CGAAGAAACCCGCTCTGCCTTTGATGTCGATTAATACTACTGCTGGT
ACGGCATCGGAAATGACACGCTTCTGTATTATCACCGATGAAGTCCG
CCACGTGAAGATGGCTATCGTCGATCGACACGTTACTCCTATGGTGT
CGGTGAATGATCCCCTCCTGATGGTCGGCATGCCCAAAGGGCTGACG
GCTGCGACGGGCATGGACGCGCTGACGCATGCTTTCGAGGCATACAG
CAGTACCGCAGCGACACCGATCACCGATGCATGTGCACTCAAGGCTG
CTAGCATGATCGCTAAAAACTTGAAGACCGCTTGTGATAACGGCAAA
GACATGCCGGCACGTGAAGCTATGGCGTACGCACAATTTCTGGCTGG
CATGGCATTCAACAATGCTAGCTTGGGCTATGTCCACGCAATGGCGC
ACCAACTGGGTGGCTATTATAACCTCCCTCACGGAGTGTGCAATGCG
GTCCTGCTGCCCCATGTTCTCGCTTACAACGCGAGTGTCGTGGCTGG
GCGTCTGAAAGATGTGGGTGTGGCAATGGGTCTGGATATTGCTAATC
TGGGCGACAAAGAAGGAGCAGAAGCTACTATCCAGGCAGTGCGGGAT
TTGGCTGCAAGTATTGGCATTCCCGCTAACCTGACCGAATTGGGCGC
GAAGAAAGAGGACGTTCCTCTGCTGGCGGATCACGCGCTCAAGGATG
CTTGCGCTCTGACAAATCCGCGCCAAGGGGATCAGAAAGAAGTCGAG
GAGCTCTTCCTGAGTGCTTTC

Ubellularia californica
FatB thioesterase
Swissprot ID: Q41635
MATTSLASAFCSMKAVMLARDGRGMKPRSSDLQLRAGNAPTSLKMING
TKFSYTESLKRLPDWSMLFAVITTIFSAAEKQWTNLEWKPKPKLPQLL
DDHFGLHGLVFRRTFAIRSYEVGPDRSTSILAVMNHMQEATLNHAKSV
GILGDGFGTTLEMSKRDLMWVVRRTHVAVERYPTWGDTVEVECWIGAS
GNNGMRRDFLVRDCKTGEILTRCTSLSVLMNTRTRRLSTIPDEVRGEI
GPAFIDNVAVKDDEIKKLQKLNDSTADYIQGGLTPRWNDLDVNQHVNN
LKYVAWVFETVPDSIFESHHISSFTLEYRRECTRDSVLRSLTTVSGGS
SEAGLVCDHLLQLEGGSEVLRARTEWRPKLTDSFRGISVIPAEPRV
(SEQ ID NO: 5; entire sequence), (bold AA are
part of expressed gene; SEQ ID NO: 9)

### Nucleic Acid Sequence:

(SEQ ID NO: 6)
ATGACAAATCTGGAATGGAAGCCTAAACCCAAGCTCCCTCAATTGTTG
GATGACCACTTCGGGCTCCATGGCCTGGTCTTTCGACGGACCTTCGCG
ATTCGGTCGTATGAAGTCGGCCCCGACCGCAGTACCAGCATTTTGGCA
GTGATGAACCACATGCAGGAAGCGACGCTGAACCACGCTAAATCCGTG
GGCATTCTCGGCGACGGTTTTGGCACGACTTTGGAGATGAGTAAACGC
GATCTCATGTGGGTGGTCCGCCGAACACACGTTGCAGTCGAACGTTAC
CCCACCTGGGGTGATACCGTCGAAGTGGAGTGCTGGATTGGCGCGAGT
GGCAATAATGGCATGCGCCGTGATTTTCTGGTTCGCGATTGCAAAACA
GGTGAAATCCTGACGCGGTGTACATCGCTGAGCGTGTTGATGAACACT
CGTACACGACGACTGTCGACTATTCCCGATGAGGTGCGCGGTGAGATC
GGGCCCGCGTTCATTGATAACGTCGCAGTCAAGGATGATGAGATCAAA
AAGCTCCAGAAACTGAATGATTCTACGGCAGATTACATCCAAGGCGGG
CTCACCCCGCGCTGGAACGATTTGGACGTGAACCAGCATGTTAATAAC
CTGAAATATGTTGCGTGGGTCTTTGAGACGGTCCCTGATTCGATTTTT
GAGAGCCACCACATCAGCTCGTTCACCTTGGAATATCGGCGGGAGTGT
ACGCGCGATTCGGTGCTGCGTAGCCTGACTACCGTCTCGGGCGGTTCC
TCCGAAGCAGGACTCGTGTGCGATCATCTCCTGCAATTGGAGGGAGGA
TCCGAGGTGCTGCGAGCTCGAACTGAATGGCGCCCAAAACTGACCGAT
AGCTTTCGGGGGATTAGCGTCATTCCCGCTGAACCGCGCGTC

Acinetobacter sp. (strain ADP1)
Wax ester synthase/acyl-CoA: diacylglycerol
acyltransferase
Swissprot ID: Q8GGG1
(SEQ ID NO: 7)
MRPLHPIDFIFLSLEKRQQPMHVGGLFLFQIPDNAPDTFIQDLVNDI
RISKSIPVPPFNNKLNGLFWDEDEEFDLDHHFRHIALPHPGRIRELL
IYISQEHSTLLDRAKPLWTCNIIEGIEGNRFAMYFKIHHAMVDGVAG
MRLIEKSLSHDVTEKSIVPPWCVEGKRAKRLREPKTGKIKKIMSGIK
SQLQATPTVIQELSQTVFKDIGRNPDHVSSFQAPCSILNQRVSSSRR
FAAQSFDLDRFRNIAKSLNVTINDVVLAVCSGALRAYLMSHNSLPSK
PLIAMVPASIRNDDSDVSNRITMILANLATHKDDPLQRLEIIRRSVQ
NSKQRFKRMTSDQILNYSAVVYGPAGLNIISGMMPKRQAFNLVISNV
PGPREPLYWNGAKLDALYPASIVLDGQALNITMTSYLDKLEVGLIAC
RNALPRMQNLLTHLEEEIQLFEGVIAKQEDIKTAN

### Nucleic Acid Sequence:

(SEQ ID NO: 8)
ATGCGACCTCTGCACCCGATTGATTTTATCTTTCTCAGTCTGGAAAA
ACGCCAACAGCCCATGCATGTCGGCGGCCTCTTTCTCTTCCAGATCC
CAGATAATGCTCCCGATACCTTCATCCAGGATTTGGTCAATGACATC
CGCATTAGCAAGAGCATTCCGGTGCCCCCATTTAACAATAAACTCAA
TGGCCTGTTTTGGGATGAAGATGAGGAATTTGATCTGGATCACCACT
TTCGACATATTGCACTGCCCCACCCGGGTCGCATCCGGGAGCTGTTG
ATTTATATCAGCCAGGAGCACTCCACTCTGCTCGATCGCGCGAAGCC
ACTGTGGACCTGCAACATTATTGAGGGCATTGAGGGCAATCGGTTCG
CTATGTACTTCAAAATCCATCACGCGATGGTTGATGGGGTCGCTGGT
ATGCGCCTGATTGAGAAGTCGTTGAGTCATGACGTTACCGAGAAATC
TATCGTCCCGCCCTGGTGCGTGGAGGGGAAGCGCGCAAAGCGGCTGC
GAGAGCCGAAAACAGGTAAAATTAAGAAAATCATGAGCGGTATCAAG
AGCCAGCTGCAAGCTACGCCAACTGTGATCCAAGAGCTGTCCCAGAC
GGTCTTTAAAGATATTGGCCGCAACCCGGACCACGTGTCCTCGTTCC
AGGCTCCTTGCTCGATCCTGAACCAACGGGTGTCTAGCTCCCGACGG
TTTGCGGCACAGTCCTTCGACTTGGATCGATTTCGCAACATCGCTAA
GAGCCTGAACGTGACAATTAACGATGTTGTGCTGGCGGTCTGCAGTG
GAGCACTCCGGGCGTACCTCATGAGCCACAATAGCCTCCCGTCTAAA
CCGTTGATCGCTATGGTGCCCGCTAGCATTCGGAACGATGACAGTGA
TGTTAGCAACCGTATTACAATGATTCTCGCTAATCTGGCAACACATA
AAGATGATCCTCTGCAGCGCTTGGAAATCATCCGCCGCTCCGTCCAA
AATTCGAAACAACGATTCAAACGCATGACCAGCGATCAGATTTTGAA
TTATTCGGCTGTTGTTTACGGCCCAGCTGGACTCAACATTATTTCTG
GCATGATGCCTAAACGGCAAGCATTTAACCTGGTGATTTCGAACGTT
CCGGGTCCGCGCGAACCGTTGTACTGGAACGGCGCGAAACTGGATGC
GCTGTACCCCGCGTCGATCGTTCTGGATGGACAGGCTCTGAACATCA
CCATGACCAGTTATTTGGATAAGCTCGAGGTGGGCTTGATTGCGTGC
CGTAATGCTCTGCCACGCATGCAGAATCTGCTGACGCACCTGGAGGA
AGAGATTCAGCTGTTCGAGGGCGTCATTGCGAAACAGGAAGATATTA
AAACGGCTAAT

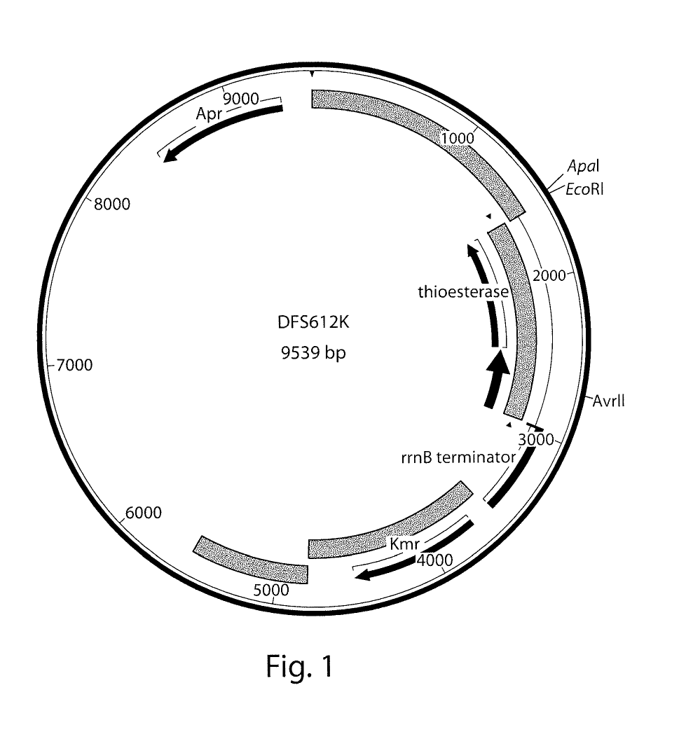

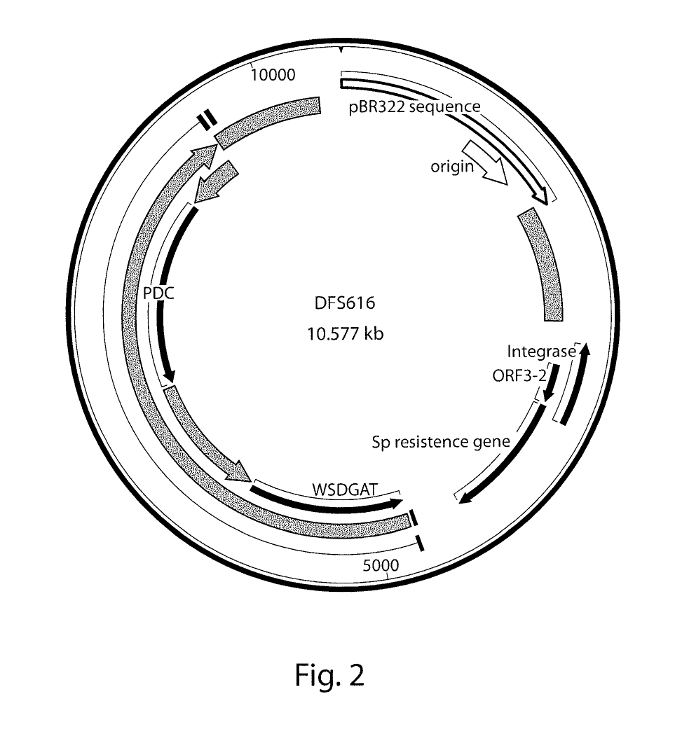

Nucleic acids encoding the enzymes are delivered to the host bacterium via one or more plasmids such as those shown in FIGS. 1-2. FIG. 1 shows a plasmid map of a construct for expression of the thioesterase enzyme, and FIG. 2 shows a plasmid map for expression of PDC, ADHE, and WSDGAT. Alternatively, all 4 genes are includes in one plasmid or in separate individual plasmids. The sequences are then incorporated into the genome of the bacterium and expressed to produce the compositions of interest.

The gene sequences and recombinant proteins described herein are isolated. The term “isolated”, as in isolated nucleic acid molecule or isolated bacterial cell, as used herein, refers to a molecule or cell that is separated from other molecules and/or cells which are present in the natural source of the molecule or cell. For example, an “isolated” nucleic acid is free of sequences which naturally flank the nucleic acid (i.e., sequences located at the 5′- and 3′-termini of the nucleic acid) in the genomic DNA of the organism from which the nucleic acid is derived. Moreover, an “isolated” nucleic acid molecule is substantially free of other cellular material, or culture medium, or of chemical precursors or other chemicals.

### A Cyanobacterial Chassis for Synthetic Biology

Synechococcus elongatus 7942 is a model organism for both circadian rhythm and photosynthesis. Synechococcus is naturally transformable and will integrate recombinant DNA into its chromosome via homologous recombination. Genes and metabolic pathways are inserted and deleted easily, and Synechococcus is an suitable platform for synthetic biology. Other suitable photosynthetic microbes (microalgae) include Gloeobacter violaceus PCC 7421, Anabaena variabilis ATCC 29413, Nostoc punctiforme PCC 73102, Nostoc sp. PCC 7120, Prochlorococcus marinus, Synechococcus elongatus PCC 6301, Synechococcus elongatus PCC 7942, Synechococcus sp. WH 8102, Synechocystis sp. PCC 6803, Thermosynechococcus elongates, Synechococcus sp. PCC7002, and Synechococcus sp. NKBG042902.

Methods were developed for culturing Synechococcus, including construction of lighted incubators and optimizing the genetic manipulations necessary for synthetic biology. Using this platform, a promoter screen was carried out to develop a library of promoters to be used for protein expression. To perform this screen, a Synechococcus codon-optimized version of the yellow fluorescent protein was used a reporter. Codons were optimized to the known natural occurrence of protein-encoding sequences in Synechococcus, e.g., codon usage website, http://www.kazusa.or.jp/codon/). By fusing promoters to this reporter, the timing (with regards to circadian rhythm) and protein expression level associated with each promoter was identified. The following exemplary promoters were identified as useful: (strong, PpplC, PapcA; medium, PrbcL; weak, Pfbp). Large fragments of DNA (˜10 kb) were integrated into the chromosome facilitating the integration of synthetic operons coding for entire metabolic pathways into the bacterium.

### Medium-Chain Fatty Acid Production in Cyanobacteria

Biodiesel is the collective term for long alkyl chain fatty acid molecules that have been esterified with a short-chain alcohol. Most often, this term refers to plant triacylglycerols that have been esterified ex vivo with methanol. Alkyl chain length has the most influence on biodiesel's chemical properties and being plant-derived biodiesel is generally longer (C16-24+) than either gasoline or petrodiesel (C6-C18). Therefore, biodiesel is slightly more energy dense than conventional petroleum fuels, but has the distinct disadvantage of a lower cloud point, the temperature at which solids begin to precipitate out of solution. This lower cloud point fundamentally limits the widespread usefulness of biodiesel. An improved biodiesel fuel is based on shorter chain fatty acids such as C12.

Fatty acid synthesis is a series of condensation reactions in which two-carbon acetyl units from acetyl-CoA are sequentially added to a growing acyl chain. Synthesis in bacteria occurs in a number of protein complexes that function as an assembly line, continually passing the growing chain down the line, until the desired length is reached and the chain is cleaved from its carrier protein by an enzyme called thioesterase. The final chain length is determined by thioesterase, which can have short, medium, or long chain specificity.

Bacterial fatty acid length is generally C16-18 and optimized for proper membrane fluidity at growth around 20-40° C. In plants, however, fatty acids are used as secondary metabolites including essential oils and waxes and chain length can vary from C6 to C24+. Due to evolutionary homology, plant and prokaryotic fatty acid synthesis pathways are structurally and functionally similar, and heterologously expressed plant enzymes, including thioesterases are functional in prokaryotes.

Expression of the California bay tree thioesterase (CBT) in E. coli results in laurate (C12) being the predominant fatty acid produced. In CBT-expressing strains, overall fatty acid content was increased nearly 5-fold, with the major component being C12. Laurate overproduction was significant enough to result in its secretion to the medium, and laurate crystals were visible on agar culture plates.

Cyanobacterial fatty acid synthesis is nearly identical that of to E. coli and results in mostly C16-C18 fatty acids. Due to this evolutionary similarity, the CBT functions similarly in Synechococcus fatty acid synthesis. A codon-optimized version of the CBT gene is used for expression in Synechococcus. Because the CBT enzyme changes lipid content, CBT constructs are made under the control of transcriptional promoters (PrbcL, PpplC, PapcA, Pfbp) of differing expression strength. These constructs are integrated into the genome using homologous recombination. Optionally, the CBT enzyme is C-terminally Streptagged for protein expression via western blots.

To analyze the effect of CBT in Synechococcus, cultures expressing CBT are grown at increasing levels (under strong, medium, or week promoters) as described above. Cultures are grown in 500 mL of BG11 medium at 30° C. under 4000 lux of illumination and diurnal light cycles using lighted incubators. Samples are collected, and growth rate and lipid content are determined in cells and medium. Growth rate is analyzed by optical absorbance at 750 nm. Lipids are extracted by standard methods, e.g., chloroformmethanol extraction, and profiled and quantitated tandem gas chromatography and mass spectrometry (GC/MS).

### Engineering a Cyanobacterium to Produce a Gasoline-Type Biofuel

Synechococcus was engineered to synthesize the medium-chain fatty acid lauric acid (C₁₁H₂₃COOH) by heterologous expression of medium-chain length specific thioesterase from the California bay tree to induce premature termination of fatty acid synthesis. In vivo biodiesel synthesis is carried out by expression of the Zymomonas mobilis ethanol fermentation pathway and the non-specific acyltransferase of Acinetobacter baylyi, which esterifies fatty acids with ethanol.

In Vivo Biodiesel Synthesis from Solar Energy

As described above, biodiesel contains of fatty acids esterified with short chain alcohols. Constructs were made to synthesize biodiesel in the organism in which fatty acids are produced from solar energy. Described herein is a complete in vivo biodiesel synthesis pathway directly from metabolites produced during photosynthesis.

Lipid-accumulating bacteria store fatty acid chains as wax esters and triaglycerols. One enzyme responsible for this process is wax ester synthase/acyl-coenzymeA:diaclyglycerol acyltransferase (WS/DGAT). The WS/DGAT gene from Acinetbobacter baylyi has a broad substrate specificity and esterifies fatty acids with a wide range of alcohols including ethanol. WS/DGAT is functional in gram-negative bacteria and in an engineered ethanol producing strain of E. coli, WS/DGAT expression resulted in the accumulation of a variety of ethyl esters. For increased biodiesel production levels, WS/DGAT is optionally co-expressed along with the CBT enzyme.

While Synechococcus does not normally produce the ethanol required for esterification, expression of the ethanol fermentation pathway from Z. mobilis results in the production of millimolar quantities of ethanol. The complete biodiesel synthesis pathway is as follows. The pyruvate decarboxylase (PDC) and alcohol dehydrogenase (ADH) genes from Z. mobilis are synthesized and codon-optimized for Synechococcus. PDC and ADH are co-expressed, and the four promoters above are tested for optimal expression level. Cultures are grown in BG11 as described above. Expression of PDC and ADH in log-phase (OD750 0.2-0.7) cultures are verified by western blot analysis. Ethanol synthesis is analyzed in a time-course fashion from log-phase to stationary phase and quantitified using standard methods, e.g., an Ethanol Enzymatic BioAnalysis kit (Roche).

The WS/DGAT gene from Acinetobacter baylyi is synthesized and codon-optimized for Synechococcus and integrated into the PDC/ADH bi-cistron for co-expression of all three enzymes. The cells are cultured using standard methods, and lipid/biodiesel is extracted using chloroformmethanol and analyzed via GC/MS. Various ethyl esters (e.g. C16 and C18) are obtained and used as standards for identification and quantitation. CBT is be incorporated using the optimal construct identified above and co-expressed with all genes.

GC/MS analysis is carried out on lipids/esters isolated to verify the shift in biodiesel length. The constructs and methods described herein produce laurate ethyl ester. High levels of laurate ethyl ester are obtained while allowing cell growth and survival.

### Example 1

### Expression of C12 and C8 Thioesterases and Production of Medium Chain-Length Fatty Acids in Cyanobacteria

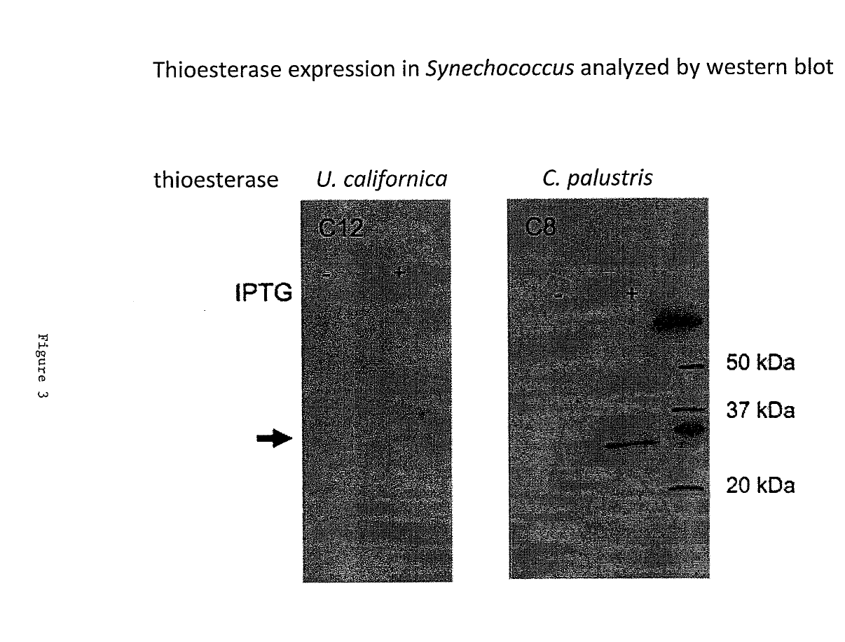

A gene encoding the C12 thioesterase from the California bay tree (U. californica) was synthesized and expressed in Synechococcus elongatus 7942 from a promoter containing the −35 region of the E. coli Trp operon promoter and the −10 region, including the lac repressor binding site. The E. coli lac repressor was also expressed in the strain from the lacI^{Q} promoter. The C12 thioesterase was expressed in with a His₆ tag and with optimized codons for expression in S. elongatus 7942. Expression of the protein was verified by Western blot using an anti-His₆ antibody as a probe (FIG. 3). Stably transformed S. elongatus 7942 with an integrated C12 thioesterase gene were induced with IPTG, and total proteins from an induced and an uninduced culture were run on SDS-PAGE, blotted to nitrocellulose, and probed with the anti-His₆ antibody. A band with the predicted molecular weight of about 30,000 Daltons was observed from the induced culture, but not from the uninduced culture.

A gene encoding a C8 thioesterase from C. palustris was expressed in Synechococcus elongatus 7942 using the same general strategy and analogous DNA constructions. Expression of the C8 thioesterase protein containing a His₆ tag was confirmed by Western blot as described above. A band of about 30,000 Daltons was observed from an IPTG-induced culture, but not from a corresponding uninduced culture.

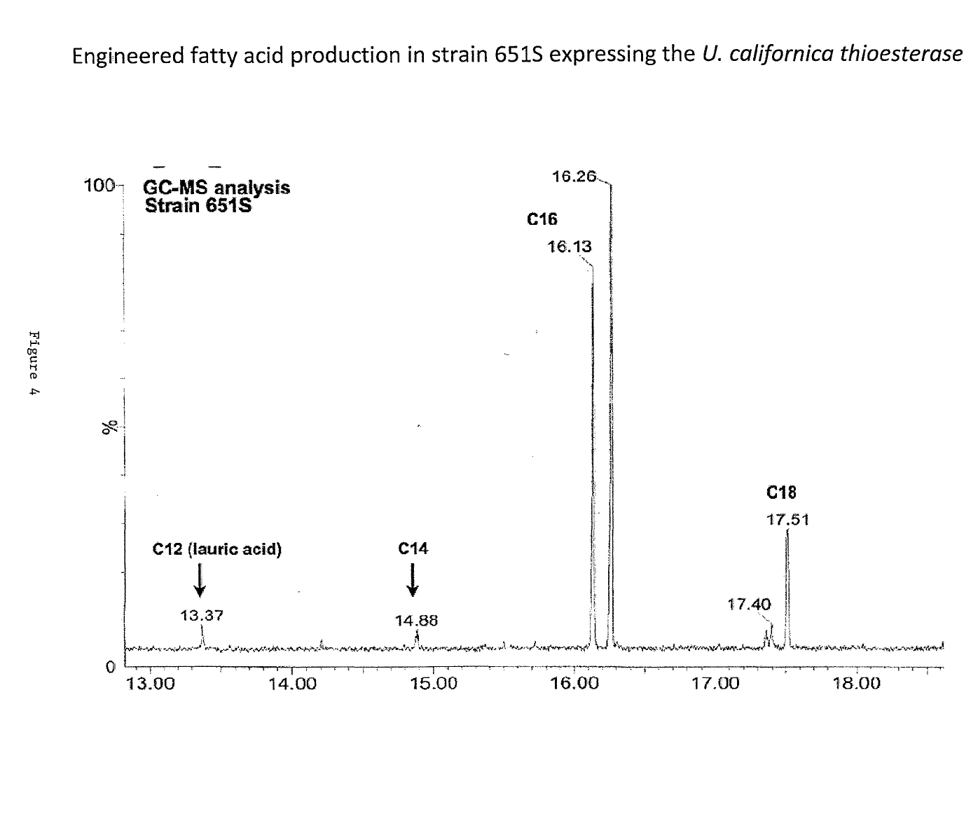

To demonstrate the production of the C12 fatty acid, i.e. lauric acid, cells were solubilized and free fatty acids were converted to the corresponding methyl esters by incubation of the cell extract in methanol according to standard procedures. This procedure is useful in preparing samples for analysis by gas chromatography-mass spectrometry. The samples were then dissolved in hexane and analyzed by gas chromatography (FIG. 4). The resulting chromatographic trace revealed the presence of both lauric acid (C12 carboxylic acid) and myristic acid (C14 carboxylic acid), which were not present in wild-type S. elongatus extracts prepared in the same manner. Mass spectrometric analysis confirmed the identity of these peaks.

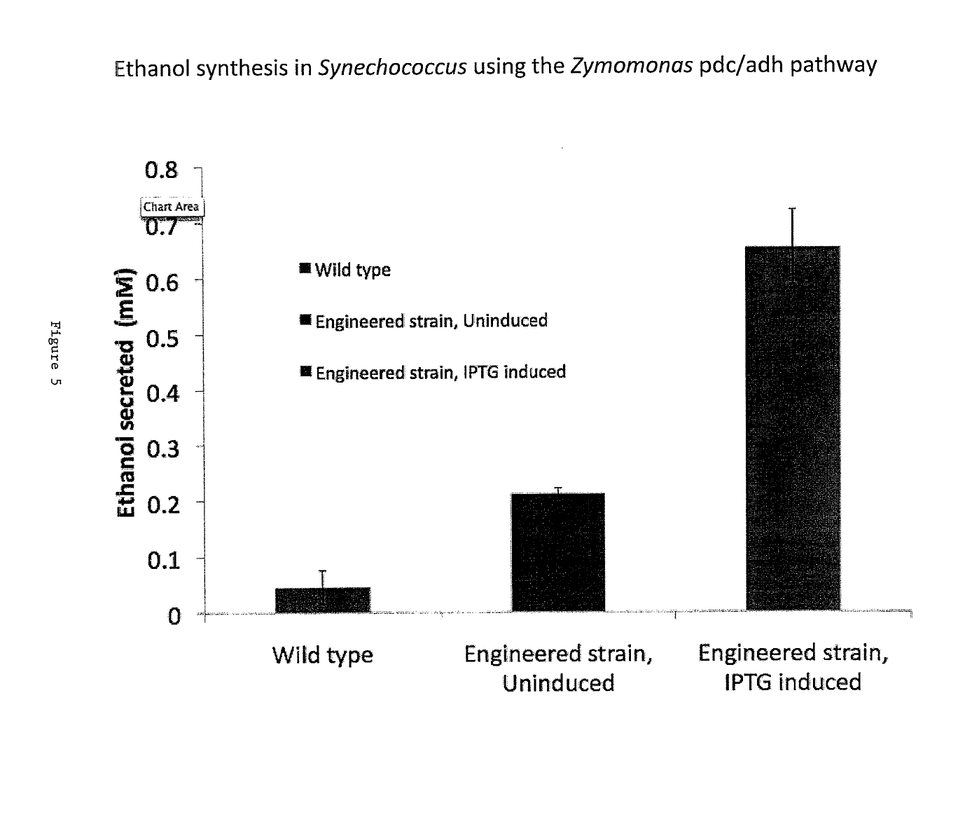

To demonstrate the production of ethanol in S. elongatus, genes from Zymomonas mobilis encoding pyruvate decarboxylase (pdc) and alcohol dehydrogenase (adh) were expressed in Synechococcus elongatus 7942 under the control of the hybrid Trp-Lac promoter described above. The lac repressor gene of E. coli was also expressed under the control of the lacI^{Q} promoter. The resulting strain was induced with IPTG and ethanol levels in the medium were measured. The induced strain produced about 675 micromolar ethanol, while under identical conditions the uninduced strain produced about 225 micromolar ethanol and the non-engineered parental Synechococcus elongatus 7942 produced only about 30 micromolar ethanol, as assayed by standard enzymatic procedures (FIG. 5).

These results indicate that medium-chain fatty acids and ethanol are produced in engineered cyanobacterial cells. According to the invention, the C12 or C8 thioesterase is co-expressed with pyruvate decarboxylase and alcohol dehydrogenase, so that both a medium chain fatty acid and ethanol are produced in the same cell. Furthermore, the C12 or C8 thioesterase is co-expressed with pyruvate decarboxylase and alcohol dehydrogenase and also with a ‘wax synthase’, with the result that the ethyl ester of a C12 or C8 fatty acid is produced. The resulting ethyl esters are purified and used as fuels.

While this invention has been particularly shown and described with references to preferred embodiments thereof, it will be understood by those skilled in the art that various changes in form and details may be made therein without departing from the scope of the invention encompassed by the appended claims.

1. An isolated photosynthetic microbe comprising a fatty acid, the length of which is less than C16.

2. The microbe of claim 1, wherein said length is less than C13.

3. The microbe of claim 1, wherein said fatty acid comprises lauric acid (C₁₁H₂₃COOH).

4. An isolated photosynthetic microbe comprising a heterologous thioesterase enzyme.

5. The microbe of claim 4, wherein said thioesterase enzyme is a California bay tree thioesterase (CBT).

6. The microbe of claim 4, wherein said thioesterase enzyme comprises the amino acid sequence of SEQ ID NO:9 or a fragment thereof.

7. The microbe of claim 4, wherein said thioesterase enzyme is encoded by the nucleic acid sequence of SEQ ID NO:6 or a fragment thereof.

8. The microbe of claim 4, wherein said cyanobacterium further comprises a heterologous wax synthase.

9. The microbe of claim 8, wherein said wax synthase comprises the amino acid sequence of SEQ ID NO:7 or a fragment thereof.

10. The microbe of claim 8, wherein said wax synthase is encoded by a nucleic acid sequence comprising SEQ ID NO:8 or a fragment thereof.

11. The microbe of claim 8, wherein said cyanobacterium further comprises a nucleic acid encoding a heterologous pyruvate decarboxylase.

12. The microbe of claim 11, wherein said pyruvate decarboxylase comprises the amino acid sequence of SEQ ID NO:1

13. The microbe of claim 11, wherein said pyruvate decarboxylase is encoded by a nucleic acid comprising SEQ ID NO:2 or a fragment thereof.

14. The microbe of claim 8, wherein said cyanobacterium further comprises a nucleic acid encoding a heterologous alcohol dehydrogenase.

15. The microbe of claim 14, wherein said alcohol dehydrogenase comprises the amino acid sequence of SEQ ID NO:3 or a fragment thereof.

16. The microbe of claim 14, wherein said pyruvate decarboxylase is encoded by a nucleic acid comprising SEQ ID NO:4 or a fragment thereof.

17. An isolated microbe comprising a nucleic acid encoding a heterologous thioesterase, wax synthase, pyruvate decarboxylase, and alcohol dehydrogenase.

18. The microbe of claim 1, wherein said microbe is a cyanobacterium.

19. The microbe of claim 1, wherein said microbe comprises a Synechococcus sp.

20. A method of producing a biodiesel fuel, comprising culturing the microbe of claim 1, extracting a medium chain fatty acid from said bacterium, and esterifying said fatty acid to yield medium chain hydrocarbon biodiesel fuel composition.

21. A method of producing a biodiesel fuel, comprising culturing the microbe of claim 17 and extracting from said bacterium a medium chain hydrocarbon biodiesel fuel composition.

22. A combustible fuel produced by the method of claim 20.
